(19) United States
(12) Patent Application Publication
(10) Pub. No.: US 2026/0054047 A1
(43) Pub. Date: Feb. 26, 2026

(54) NEEDLELESS CONNECTOR WITH TEAR-RESISTANT SEAL

(71) Applicant: CAREFUSION 303, INC., San Diego, CA (US)

(72) Inventors: Leah Paige GAFFNEY, Orange, CA (US); Christopher Jesse ZOLLINGER, Chino, CA (US)

(21) Appl. No.: 19/307,663

(22) Filed: Aug. 22, 2025

Related U.S. Application Data
(60) Provisional application No. 63/686,669, filed on Aug. 23, 2024.

(51) Int. Cl. A61M 39/04; A61M 5/14; A61M 25/00; A61M 39/00; A61M 39/02

(52) U.S. Cl. CPC A61M 39/045; A61M 5/1413; A61M 25/0097; A61M 2039/0036; A61M 2039/027; A61M 2207/00

(57) ABSTRACT

Seals for medical fluid connectors are disclosed. A seal as molded may have an elliptical cross-sectional shape, an aperture on the top face of the seal, a well inside the seal, and a divet on the bottom face of the seal. The seal as molded becomes assembled when it is constrained by a housing. When constrained by the housing, the seal has a circular cross-sectional shape, and the aperture becomes an elongate insertion area. The elongate insertion area is closed such that fluid cannot permeate the seal as assembled. However, the seal as assembled is penetrable by a center post through the elongate insertion area, and the center post creates a fluid path through the seal when the center post penetrates the seal.

### RELATED APPLICATIONS

This application claims priority to U.S. Provisional Application No. 63/686,669, which was filed on Aug. 23, 2024, and is entitled “NEEDLE FREE CONNECTOR WITH TEAR-RESISTANT SEAL,” the entire disclosure of which is hereby incorporated by reference.

### TECHNICAL FIELD

The present disclosure relates generally to medical fluid connectors and, more particularly, to neutral displacement needle-free connectors that reduce the occurrence of dislodgement of an intravenous (IV) catheter that is connected to a patient.

### BACKGROUND

Medical treatments often include the infusion of a medical fluid (e.g., a saline solution or a liquid medication) to patients using an IV catheter that is connected through an arrangement of flexible tubing and fittings, commonly referred to as an “IV set,” to a source of fluid, for example an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

Manufacturing medical fluid connectors and, more particularly, neutral displacement needle-free connectors often requires use of razors to cut slits in the seals of such connectors.

### SUMMARY

In accordance with at least some embodiments disclosed herein is the realization that components used with neutral fluid displacement, such as a luer and a post, can break when overlapping and contacting each other, which can result in medical fluid leakage and reduced medical delivery to a patient. When a broken component is inside a connector body, it may not be readily apparent to a medical professional that the component is broken, leading to delayed response times.

Aspects of the present disclosure provide a needle-free fluid connector assembly that provides a neutral fluid displacement connection while minimizing the risk of breaking one or more components of the needle-free fluid connector assembly. The neutral fluid displacement limits or prevents fluid from entering a catheter lumen during connection or disconnection of a medical fluid.

In accordance with at least some embodiments disclosed herein is the realization that the process of manufacturing medical fluid connectors—more particularly, the use of razors to cut slits in the seals of such connectors—can result in inconsistent connectors. Additionally, the use of razors to cut slits in the seals of medical fluid connectors can create corners with high stress conditions that are susceptible to tearing. Such tearing can decrease the ability of the connectors to properly seal.

Aspects of the present disclosure provide a medical fluid connector that is manufactured without the use of razors. Molding, compression, and/or lubrication are used to manufacture elongate insertion areas that serve the same purpose as the slits in traditional medical fluid connectors.

Accordingly, aspects of the present disclosure provide a seal for a needleless fluid connector, comprising: a top end, a bottom end, a longitudinal axis extending between the top and bottom ends of the seal, and an aperture extending through the seal along the longitudinal axis; an elliptical cross-sectional shape having a long axis and a short axis, both transverse to the longitudinal axis, when the seal is unconstrained by the needleless fluid connector; a circular cross-sectional shape when the seal is compressed, by positioning the seal within the needleless fluid connector, along the long axis such that the long axis is substantially equal to the short axis; an elongate insertion area on the top end, formed from the aperture when the seal is compressed along the long axis, that restricts fluid communication through the seal; and a well on an inner surface of the aperture, the well configured to restrict friction by (a) limiting surface area contact between the seal and a penetrating member extending through the aperture and (b) containing a lubricant.

In some embodiments, the housing has an inner volume with a circular cross-section, and the housing compresses the seal until the seal transforms from its initial elliptical cross-section to its final circular cross-section.

In some embodiments, the insertion area comprises an area of decreased tension that is formed when the aperture is compressed by the housing.

In some embodiments, wherein a divet is located on the bottom end of the seal and is centered on the longitudinal axis.

In some embodiments, the divet comprises a hollowed portion of the seal that limits the friction between the seal and the penetrating member.

In some embodiments, the well is located between the insertion area and the divet along the longitudinal axis.

In some embodiments, the lubricant in the well limits the amount of friction between the penetrating member and the seal when the penetrating member penetrates the seal.

In some embodiments, the penetrating member creates a fluid path through the seal when the penetrating member penetrates the seal.

Aspects of the present disclosure also provide a method for manufacturing a seal for a needleless fluid connector, the method comprising: molding a seal with a top end, a bottom end, and a longitudinal axis that extends between the top and bottom ends, wherein the seal has an elliptical cross-sectional shape; molding an aperture on the top end of the seal; molding a well on an inner surface of the aperture to restrict friction by limiting surface area contact between the seal and a penetrating member extending through the aperture; applying a lubricant to the well to further restrict friction between the seal and the penetrating member; and compressing the seal in a housing until the seal has a circular cross-sectional shape and the aperture forms an insertion area through which a fluid cannot pass.

In some embodiments, the insertion area comprises an area of decreased tension that is formed when the aperture is compressed by the housing.

In some embodiments, a divet is located on a bottom end of the seal and is centered on the longitudinal axis.

In some embodiments, the divet comprises a hollowed portion of the seal that restricts friction between the center post and the seal by limiting surface area contact between the center post and the seal.

In some embodiments, the well is located between the insertion area and the divet along the longitudinal axis.

In some embodiments, the center post can penetrate the seal through the insertion area, and the center post creates a fluid path through the seal when the center post penetrates the seal.

Accordingly, the present application addresses several operational challenges encountered in prior neutral displacement connector assemblies that are susceptible to breaking or dislodging from the patient.

The present application also addresses manufacturing and operational challenges encountered in prior medical fluid connectors that are susceptible to manufacturing inconsistencies and premature tearing.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a cross-sectional view of the medical fluid connector in FIG. 1, in accordance with aspects of the present disclosure.

FIG. 3 illustrates a perspective view of the seal as assembled, in accordance with aspects of the present disclosure.

FIG. 4 illustrates a perspective view of the seal as molded, in accordance with aspects of the present disclosure.

FIG. 5 illustrates a cross-sectional view of the seal as molded, in accordance with aspects of the present disclosure.

FIG. 6 illustrates a flowchart showing a method for manufacturing a seal for a needleless fluid connector.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of manufacturing medical fluid connectors without the use of razors, thus minimizing the likelihood of manufacturing inconsistent connectors and forming corners with high stress conditions that are susceptible to tearing.

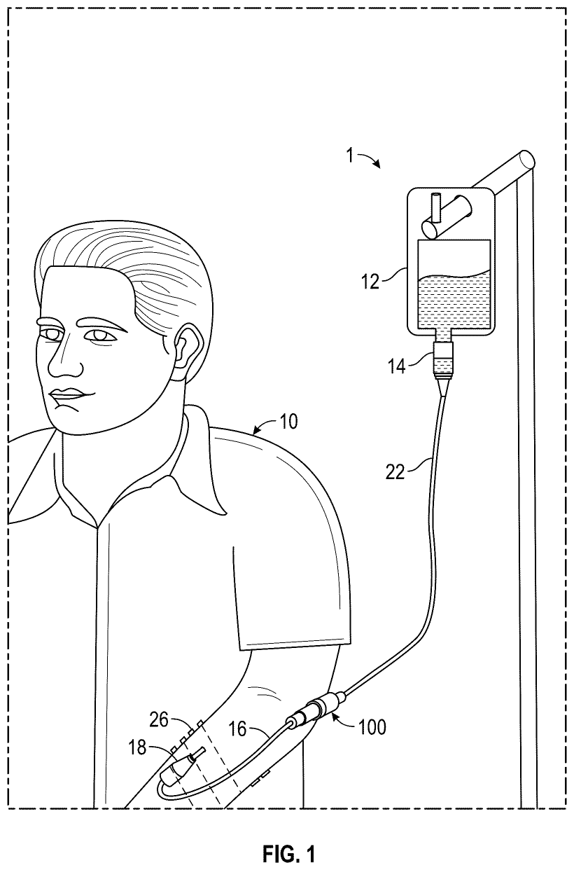

Referring now to the figures, FIG. 1 illustrates an IV set 1 coupled to a patient 10, in accordance with the aspects of the present disclosure. The IV set 1 includes a medical fluid bag 12, a drip chamber 14, and tubing 22. The tubing 22 extends between the drip chamber 14 and a medical fluid connector 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

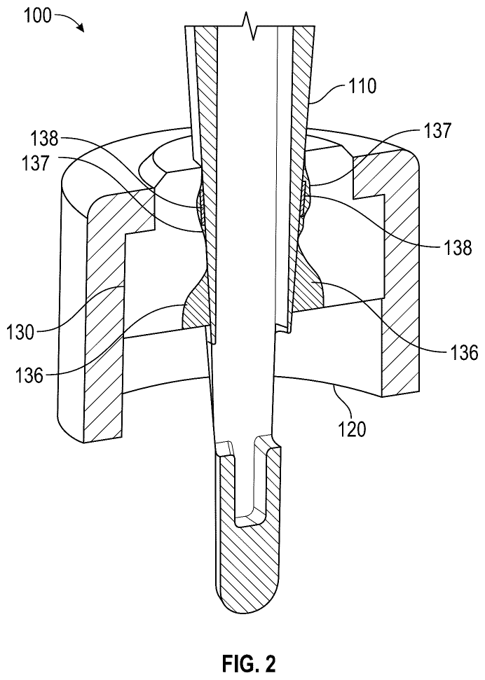

FIG. 2 illustrates a cross-sectional view of the medical fluid connector 100 for use with an IV set, in accordance with the aspects of the present disclosure. The medical fluid connector 100 is designed for use in medical applications, such as the IV set 1 as well as other IV medical fluid delivery applications using catheters, including peripheral intravenous catheters (PIVC), as non-limiting examples.

The medical fluid connector 100 provides a fluid path. As shown, the medical fluid connector 100 includes a penetrating member 110, a housing 120, and a seal 130. In some embodiments, the penetrating member 110 is a center post. The center post 110 penetrates the seal 130 to create the fluid path through the seal 130. The housing 120 has an inner volume with a circular cross-section.

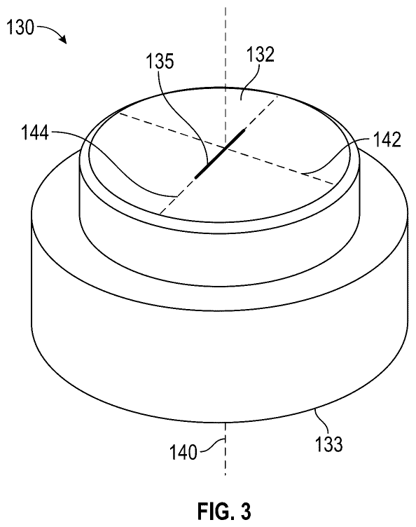

FIG. 3 illustrates a perspective view of the seal 130 as assembled. The seal 130 as assembled is constrained and/or compressed by the housing 120 and has a circular cross-sectional shape. In other words, the long axis 142 is substantially the same length as the short axis 144. The seal 130 includes a top end 132 and a bottom end 133. The diameter of the seal 130 can vary between the top end 132 and the bottom end 133. In some embodiments of the assembled seal 130, the diameter of the top portion of the seal 130 is shorter than the diameter of the bottom portion of the seal 130. The seal 130 includes an elongate insertion area 135 on the top end 132 along the longitudinal axis 140. Fluid cannot permeate the elongate insertion area 135. However, the elongate insertion area 135 creates an area of decreased tension through which a center post 110 (shown in FIG. 2) can penetrate the seal 130 and create a fluid path through the seal 130.

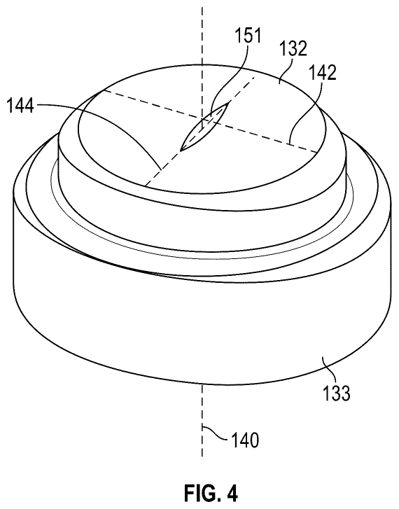

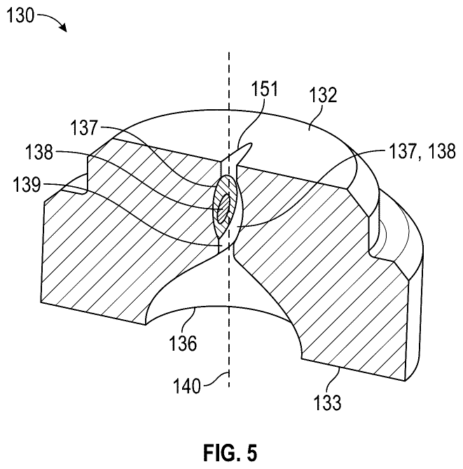

FIG. 4 illustrates a perspective view of the seal as molded. The seal 130 as molded is not constrained by the housing 120 and may have an elliptical cross-sectional shape. In other words, the long axis 142 (i.e., the semi-major axis of the elliptical cross-sectional shape) is longer than the short axis 144 (i.e., the semi-minor axis of the elliptical cross-sectional shape). In some embodiments, the short axis 144 is 10% to 40% of the length of the long axis 142. In a preferred embodiment, the short axis 144 is 10% to 20% of the length of the long axis 142. The long axis 142 and short axis 144 of the seal 130 can vary between the top end 132 and the bottom end 133. In some embodiments of the seal 130 as molded, the long axis 142 and short axis 144 of the top portion of the seal 130 are shorter than the long axis 142 and the short axis 144 of the bottom portion of the seal 130. In some embodiments, the seal 130 as molded has beveled edges. As molded, the seal 130 may include an aperture 151 on the top end 132 of the along the longitudinal axis 140. The aperture 151 may have an elliptical cross-sectional shape. In some embodiments, the semi-major axis of the elliptical aperture is perpendicular to the direction of the compression. The aperture 151 may have a depth that is up to one-half the depth of the seal 130. The aperture 151 has an inner surface 139 (shown in FIG. 5).

The seal 130 is assembled when the seal 130 is constrained by the housing 120. The housing 120 compresses the seal 130 until the elliptical cross-sectional shape becomes the circular cross-sectional shape. In other words, the seal 130 is assembled when the long axis 142 is substantially the same length as the short axis 144. The height (i.e., the distance between the top end 132 and the bottom end 133) of the seal 130 as molded in FIG. 4 is substantially the same as the height of the seal 130 as assembled in FIG. 3. The compression may close the aperture 151 and transform the aperture 151 into the elongate insertion area 135. The elongate insertion area 135 (also referred to as the insertion area) comprises an area of decreased tension through which the center post 110 can penetrate the seal 130. Because of the presence of the aperture 151 on the seal 130 as molded, the less force is applied to the center post 110 at the insertion area 135 than would be applied in the absence of the aperture 151. This is because the presence of the aperture 151 requires that the seal 130 has less material at the location of the aperture 151 than the seal 130 would have had in the absence of the aperture 151. Because there is less material on the seal 130 as molded at the location of the aperture 151, the seal 130 as assembled applies less force to the center post 110 at the insertion area 135.

FIG. 5 illustrates a cross-sectional view of the seal as molded. The seal 130 may include a divet 136 on the bottom end 133 of the seal 130 along the longitudinal axis 140. The divet 136 may have a generally conical shape, but other shapes are also possible. The vertex of the divet 136 may point towards the top end 132 of the seal 130. The base of the divet 136 may have a diameter that is between one-fifth and one-third of the diameter of the bottom end of the seal. The divet 136 may have a height that is between one-tenth and one-half of the height of the seal 130. The divet 136 reduces the amount of friction between the center post 110 and the seal 130 by reducing the contact area between the center post 110 and the seal 130. Due to the presence of the divet 136, the center post 110 does not necessarily engage with the entirety of the inner surface 139.

On the inner surface 139, along the longitudinal axis 140, there may be a well 137. The well 137 may be located between the aperture 151 and the divet 136. The well 137 restricts friction between the center post 110 and the seal 130 by limiting the surface area contact between the center post 110 and the seal 130. The well 137 may include a lubricant 138 that further limits the amount of friction between the center post 110 and the seal 130.

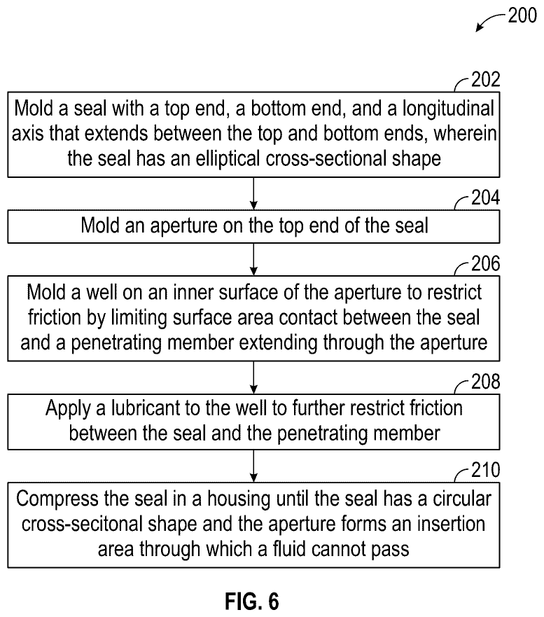

FIG. 6 illustrates a flowchart 200 showing a method for manufacturing a seal for a needleless medical fluid connector, in accordance with aspects of the present disclosure. Seals for needleless medical fluid connectors shown or described herein are manufactured by carrying out the steps of the method shown in the flowchart 200.

In step 202, a seal with a top end, a bottom end, and a longitudinal axis is molded. The longitudinal axis extends between the top and bottom ends. The seal has an elliptical cross-sectional shape as molded.

In step 204, an aperture is molded on a top end of the seal. The aperture may be located on a longitudinal axis of the elliptical prism. The aperture may have an elliptical cross-sectional shape.

In step 206, a well is molded on an inner surface of the seal. The well may be located on the longitudinal axis of the elliptical prism. The well may be located in between the aperture on the top end of the elliptical prism and a divet on a bottom end of the elliptical prism. The well may restrict friction between a penetrating member (e.g., a center post) that penetrates the seal and the seal by limiting the surface area contact between the seal and the penetrating member. In some embodiments, a divet is molded beneath the aperture. The divet is located on a bottom end of the seal and is centered on the longitudinal axis. The divet is a hollowed portion of the seal that restricts friction between the center post and the seal by limiting surface area contact between the center post and the seal.

In step 208, lubricant is applied to the well. The lubricant in the well restricts friction between the seal and the penetrating member.

In step 210, the seal is compressed in a housing until the seal has a circular cross-sectional shape and the aperture forms an insertion area. The seal may be compressed into a generally cylindrical shape. Fluid cannot permeate the elongate insertion area. However, the elongate insertion area creates an area of decreased tension through which a center post can penetrate the seal. When the center post penetrates the seal, the center post creates a fluid path through the seal.

### Illustration of Subject Technology As Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A seal for a needleless fluid connector, comprising: a top end, a bottom end, a longitudinal axis extending between the top and bottom ends of the seal, and an aperture extending through the seal along the longitudinal axis; an elliptical cross-sectional shape having a long axis and a short axis, both transverse to the longitudinal axis, when the seal is unconstrained by the needleless fluid connector; a circular cross-sectional shape when the seal is compressed, by positioning the seal within the needleless fluid connector, along the long axis such that the long axis is substantially equal to the short axis; an elongate insertion area on the top end, formed from the aperture when the seal is compressed along the long axis, that restricts fluid communication through the seal; and a well on an inner surface of the aperture, the well configured to restrict friction by (a) limiting surface area contact between the seal and a penetrating member extending through the aperture and (b) containing a lubricant.

Clause 2. The seal of clause 1, wherein the housing has an inner volume with a circular cross-section, and the housing compresses the seal until the seal transforms from its initial elliptical cross-section to its final circular cross-section.

Clause 3. The seal of clause 1, wherein the insertion area comprises an area of decreased tension that is formed when the aperture is compressed by the housing.

Clause 4. The seal of clause 1, wherein a divet is located on the bottom end of the seal and is centered on the longitudinal axis.

Clause 5. The seal of clause 4, wherein the divet comprises a hollowed portion of the seal that limits the friction between the seal and the penetrating member.

Clause 6. The seal of clause 4, wherein the divet comprises a height that is up to one-half of a height of the seal.

Clause 7. The seal of clause 4, wherein the divet comprises a base area that occupies up to one-third of an area the bottom end of the seal.

Clause 8. The seal of clause 4, wherein the well is located between the insertion area and the divet along the longitudinal axis.

Clause 9. The seal of clause 1, wherein the lubricant in the well limits the amount of friction between the penetrating member and the seal when the penetrating member penetrates the seal.

Clause 10. The seal of clause 1, wherein the penetrating member creates a fluid path through the seal when the penetrating member penetrates the seal.

Clause 11. A seal for a needleless fluid connector, comprising: a top end, a bottom end, and a longitudinal axis extending between the top and bottom ends of the seal; an elongate insertion area on the top end that restricts fluid communication through the seal; a well inside of the seal, the well configured to restrict friction by (a) limiting surface area contact between the seal and a penetrating member extending through the elongate insertion area and (b) containing a lubricant; and a divet positioned on the bottom end of the seal and symmetrical about the longitudinal axis, the seal configured to restrict friction by limiting surface area contact between the seal and a penetrating member extending through the elongate insertion area.

Clause 12. The seal of clause 11, wherein the divet comprises a height that is up to one-half of a height of the seal.

Clause 13. The seal of clause 11, wherein the divet comprises a base area that occupies up to one-third of an area the bottom end of the seal.

Clause 14. A method for manufacturing a seal for a needleless fluid connector, the method comprising: molding a seal with a top end, a bottom end, and a longitudinal axis that extends between the top and bottom ends, wherein the seal has an elliptical cross-sectional shape;
molding an aperture on the top end of the seal; molding a well on an inner surface of the aperture to restrict friction by limiting surface area contact between the seal and a penetrating member extending through the aperture; applying a lubricant to the well to further restrict friction between the seal and the penetrating member; and compressing the seal in a housing until the seal has a circular cross-sectional shape and the aperture forms an insertion area through which a fluid cannot pass.

Clause 15. The method of clause 14, wherein the insertion area comprises an area of decreased tension that is formed when the aperture is compressed by the housing.

Clause 16. The method of clause 14, wherein a divet is located on a bottom end of the seal and is centered on the longitudinal axis.

Clause 17. The method of clause 14, wherein the divet comprises a hollowed portion of the seal that restricts friction between the center post and the seal by limiting surface area contact between the center post and the seal.

Clause 18. The method of clause 14, wherein the divet comprises a height that is up to one-half of a height of the seal and a base area that occupies up to one-third of an area the bottom end of the seal.

Clause 19. The method of clause 14, wherein the well is located between the insertion area and the divet along the longitudinal axis.

Clause 20. The method of clause 14, wherein the center post can penetrate the seal through the insertion area, and the center post creates a fluid path through the seal when the center post penetrates the seal.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more. ” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration. ” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled”or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims.

Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for. ” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A seal for a needleless fluid connector, comprising:
a top end, a bottom end, a longitudinal axis extending between the top and bottom ends of the seal, and an aperture extending through the seal along the longitudinal axis;
an elliptical cross-sectional shape having a long axis and a short axis, both transverse to the longitudinal axis, when the seal is unconstrained by the needleless fluid connector;
a circular cross-sectional shape when the seal is compressed, by positioning the seal within the needleless fluid connector, along the long axis such that the long axis is substantially equal to the short axis;
an elongate insertion area on the top end, formed from the aperture when the seal is compressed along the long axis, that restricts fluid communication through the seal; and
a well on an inner surface of the aperture, the well configured to restrict friction by (a) limiting surface area contact between the seal and a penetrating member extending through the aperture and (b) containing a lubricant.

2. The seal of claim 1, wherein the housing has an inner volume with a circular cross-section, and the housing compresses the seal until the seal transforms from its initial elliptical cross-section to its final circular cross-section.

3. The seal of claim 1, wherein the insertion area comprises an area of decreased tension that is formed when the aperture is compressed by the housing.

4. The seal of claim 1, wherein a divet is located on the bottom end of the seal and is centered on the longitudinal axis.

5. The seal of claim 4, wherein the divet comprises a hollowed portion of the seal that limits the friction between the seal and the penetrating member.

6. The seal of claim 4, wherein the divet comprises a height that is up to one-half of a height of the seal.

7. The seal of claim 4, wherein the divet comprises a base area that occupies up to one-third of an area the bottom end of the seal.

8. The seal of claim 4, wherein the well is located between the insertion area and the divet along the longitudinal axis.

9. The seal of claim 1, wherein the lubricant in the well limits the amount of friction between the penetrating member and the seal when the penetrating member penetrates the seal.

10. The seal of claim 1, wherein the penetrating member creates a fluid path through the seal when the penetrating member penetrates the seal.

11. A seal for a needleless fluid connector, comprising:
a top end, a bottom end, and a longitudinal axis extending between the top and bottom ends of the seal;
an elongate insertion area on the top end that restricts fluid communication through the seal;
a well inside of the seal, the well configured to restrict friction by (a) limiting surface area contact between the seal and a penetrating member extending through the elongate insertion area and (b) containing a lubricant; and
a divet positioned on the bottom end of the seal and symmetrical about the longitudinal axis, the seal configured to restrict friction by limiting surface area contact between the seal and a penetrating member extending through the elongate insertion area.

12. The seal of claim 11, wherein the divet comprises a height that is up to one-half of a height of the seal.

13. The seal of claim 11, wherein the divet comprises a base area that occupies up to one-third of an area the bottom end of the seal.

14. A method for manufacturing a seal for a needleless fluid connector, the method comprising:
molding a seal with a top end, a bottom end, and a longitudinal axis that extends between the top and bottom ends, wherein the seal has an elliptical cross-sectional shape;
molding an aperture on the top end of the seal;
molding a well on an inner surface of the aperture to restrict friction by limiting surface area contact between the seal and a penetrating member extending through the aperture;
applying a lubricant to the well to further restrict friction between the seal and the penetrating member; and
compressing the seal in a housing until the seal has a circular cross-sectional shape and the aperture forms an insertion area through which a fluid cannot pass.

15. The method of claim 14, wherein the insertion area comprises an area of decreased tension that is formed when the aperture is compressed by the housing.

16. The method of claim 14, wherein a divet is located on a bottom end of the seal and is centered on the longitudinal axis.

17. The method of claim 14, wherein the divet comprises a hollowed portion of the seal that restricts friction between the center post and the seal by limiting surface area contact between the center post and the seal.

18. The method of claim 14, wherein the divet comprises a height that is up to one-half of a height of the seal and a base area that occupies up to one-third of an area the bottom end of the seal.

19. The method of claim 14, wherein the well is located between the insertion area and the divet along the longitudinal axis.

20. The method of claim 14, wherein the center post can penetrate the seal through the insertion area, and the center post creates a fluid path through the seal when the center post penetrates the seal.
